(19) United States
(12) Patent Application Publication
(10) Pub. No.: US 2022/0072022 A1
(43) Pub. Date: Mar. 10, 2022

(54) ANTI-AGING AGENT OR LIFE-EXTENDING AGENT

(71) Applicant: StaGen Co., Ltd., tokyo (JP)

(72) Inventors: Shohei MITANI, Tokyo (JP); Sawako MOIZUMI, Tokyo (JP); Luna IZUHARA, Tokyo (JP); Naoyuki KAMATANI, Tokyo (JP)

(73) Assignee: StaGen Co., Ltd., Tokyo (JP)

(21) Appl. No.: 17/422,347

(22) PCT Filed: Jan. 10, 2020
(86) PCT No.: PCT/JP2020/000551

§ 371 Date: Jul. 12, 2021

(30) Foreign Application Priority Data
Jan. 18, 2019 (JP) 2019-006871

(51) Int. Cl. A61K 31/7076; A61K 31/522; A61K 31/426; A61K 31/519; A61K 31/444; A61K 31/405; A61K 31/415; A61K 31/708; A61P 39/00

(52) U.S. Cl. CPC A61K 31/7076; A61K 31/522; A61K 31/426; A61K 31/519; A61P 39/00; A61K 31/405; A61K 31/415; A61K 31/708; A61K 31/444

(57) ABSTRACT

An object of the present invention is to enable prevention of aging and extension of lifespan of a human or non-human organism by a method other than calorie restriction and administration of metformin.

It is possible to prevent aging and extend the lifespan of a human or non-human organism by administering an agent comprising A) a xanthine oxidase/xanthine dehydrogenase inhibitor.

In addition, it is possible to prevent aging and extend the lifespan of a human or non-human organism by combining A) a xanthine oxidase/xanthine dehydrogenase inhibitor and B) hypoxanthine, or a compound that is capable of being converted to hypoxanthine in body, by administering these simultaneously or administering these as a combination drug or a kit formulation.

### TECHNICAL FIELD

The present invention relates to an agent for suppressing aging (advancement in age) of humans or non-human organisms and extending their lifespan. More specifically, the present invention relates to an agent for suppressing aging and extending lifespan by administering, to humans or non-human organisms, A) a xanthine oxidase/xanthine dehydrogenase inhibitor alone, or a combination of A) xanthine oxidase/xanthine dehydrogenase inhibitor and B) hypoxanthine, or a compound that is capable of being converted to hypoxanthine in the body, such as inosine.

### BACKGROUND ART

Aging is biological changes that occur in an individual over time. Among the changes, in particular, aging refers to various functional declines and processes thereof that occur before the death of an organism. In humans, aging is often characterized by skin wrinkles, muscle weakness, hearing loss, poor vision, and neurological disorders such as dementia. It has been reported that calorie restriction reduces aging and extends lifespan (Non-Patent Document 1), and suppression of aging may lead to prolongation of lifespan. However, there is no known cure that reliably prevents human aging and prolongs lifespan. Anyway, various factors have been reported to be involved in aging and longevity. Among them, it has been suggested that energy decrease due to mitochondrial dysfunction or functional disorder of mitochondria is the mechanism of aging (Non-Patent Documents 2 to 7).

It is known that the number and function of mitochondria decrease by 8% for every 10 years of aging in humans (Non-Patent Document 8), and in the elderly, mitochondrial function may not be sufficient to support normal functions of a living body. Mitochondrial function is to produce ATP, which is energy, and thus a decrease in mitochondrial function leads to a decrease in cellular energy, which may accelerate aging. Mitochondrial disease is a general term for diseases in which disorders occur in mitochondria due to genes (Non-Patent Documents 5 to 7). Mitochondrial disease causes hearing loss, brain disorder, muscle disorder, cardiomyopathy, and diabetes, which are similar to those disorders caused by aging.

It has already been reported that by using a high dose of a xanthine oxidase/xanthine dehydrogenase inhibitor such as allopurinol and febuxostat, cardiac disorders (Non-Patent Document 9), muscle atrophy (Non-Patent Document 10), and incident dementia (Non-Patent Document 11) are suppressed.

### CITATION LIST

### Non-Patent Literature

Non-Patent Document 1: Taylor R C, DillinA (2011). “Aging as an event of proteostasis collapse”. Cold Spring Harb Perspect Biol. 3 (5): a004440.
Non-Patent Document 2: Srivastava S. The Mitochondrial Basis of Aging and Age-Related Disorders. Genes (Basel). 2017 Dec. 19; 8(12). pii: E398.
Non-Patent Document 3: Sun N, Youle R J, Finkel T. The Mitochondrial Basis of Aging. Mol Cell. 2016 Mar. 3; 61(5):654-666.
Non-Patent Document 4: Wallace, D. C. (2005) A mitochondrial paradigm of metabolic and degenerative diseases, aging, and cancer: a dawn for evolutionary medicine. Annu. Rev. Genet. 39, 359-407
Non-Patent Document 5: Pfeffer, G., Majamaa, K., Turnbull, D. M., Thorburn, D. and Chinnery, P. F. (2012)
Non-Patent Document 6: Viscomi C. Toward a therapy for mitochondrial disease. Biochem Soc Trans. 2016 Oct. 15; 44(5):1483-1490.
Non-Patent Document 7: Alston C L, Rocha M C, Lax N Z, Turnbull D M, Taylor R W. The genetics and pathology of mitochondrial disease. J Pathol. 2017 January; 241(2):236-250.
Non-Patent Document 8: Short K R, Bigelow M L, Kahl J, Singh R, Coenen-Schimke J, Raghavakaimal S, Nair K S. Decline in skeletal muscle mitochondrial function with aging in humans. Proc Natl Acad Sci USA. 2005 Apr. 12; 102(15):5618-23.
Non-Patent Document 9: Noman A, Ang D S, Ogston S, Lang C C, Struthers A D. Effect of high-dose allopurinol on exercise in patients with chronic stable angina: a randomized placebo controlled crossover trial. Lancet. 2010 Jun. 19; 375(9732):2161-7.
Non-Patent Document 10: Ferrando B, Gomez-Cabrera M C, Salvador-Pascual A, Puchades C, Derbre F, Gratas-Delamarche A, Laparre L, Olaso-Gonzalez G, Cerda M, Viosca E, Alabajos A, Sebastia V, Alberich-Bayarri A, Garcia-Castro F, Vina J. Allopurinol partially prevents disuse muscle atrophy in mice and humans. Sci Rep. 2018 February
Non-Patent Document 11: Singh J A, Cleveland J D. Comparative effectiveness of allopurinol versus febuxostat for preventing incident dementia in older adults: a propensity-matched analysis. Arthritis Res Ther. 2018 Aug. 3; 20(1):167.
Non-Patent Document 12: Kamatani N, Hashimoto M, Sakurai K, Gokita K, Yoshihara J, Sekine M, Mochii M, Fukuuchi T, Yamaoka N, Kaneko K. Clinical studies on changes in purine compounds in blood and urine by the simultaneous administration of febuxostat and inosine, or by single administration of each. Gout and Nucleic Acid Metabolism 41, 171-181, 2017.
Non-Patent Document 13: Onken B, Driscoll M. Metformin induces a dietary restriction-like state and the oxidative stress response to extend C. elegans Healthspan via AMPK, LKB1, and SKN-1. PLoS One. 2010 Jan. 18; 5(1):e8758.
Non-Patent Document 14: Cabreiro F, Au C, Leung K Y, Vergara-Irigaray N, Cocheme H M, Noori T, Weinkove D, Schuster E, Greene N D, Gems D. Metformin retards aging in C. elegans by altering microbial folate and methionine metabolism. Cell. 2013 Mar. 28; 153(1):228-39.
Non-Patent Document 15: De Haes W, Frooninckx L, Van Assche R, Smolders A, Depuydt G, Billen J, Braeckman B P, Schoofs L, Temmerman L. Metformin promotes lifespan through mitohormesis via the peroxiredoxin PRDX-2. Proc Natl Acad Sci USA. 2014 Jun. 17; 111(24):E2501-9.
Non-Patent Document 16: Chen J, Ou Y, Li Y, Hu S, Shao L W, Liu Y. Metformin extends C. elegans lifespan through lysosomal pathway. Elife. 2017 Oct. 13; 6. pii: e31268.
Non-Patent Document 17: Na H J, Pyo J H, Jeon H J, Park J S, Chung H Y, Yoo M A. Deficiency of Atg6 impairs beneficial effect of metformin on intestinal stem cell aging in Drosophila. Biochem Biophys Res Commun. 2018 Mar. 25; 498 (1):18-24.
Non-Patent Document 18: Campbell J M, Bellman S M, Stephenson M D, Lisy K. Metformin reduces all-cause mortality and diseases of ageing independent of its effect on diabetes control: A systematic review and meta-analysis. Ageing Res Rev. 2017 November; 40:31-44.
Non-Patent Document 19: Kleiber, M. Body Size and Metabolism. HILGARDIA 6, (1932).
Non-Patent Document 20: Brody, S. Bioenergetics and Growth (Hafner Publishing Co., New York, 1945).
Non-Patent Document 21: Fox I H, Palella T D, Kelley W N. Hyperuricemia: a marker for cell energy crisis. N Engl J Med 317, 111-112, 1987
Non-Patent Document 22: Hosoyamada M, Tsurumi Y, Hirano H, Tomioka N H, Sekine Y, Morisaki T, Uchida S. Uratl-Uox double knockout mice are experimental animal models of renal hypouricemia and exercise-induced acute kidney injury. Nucleosides Nucleotides Nucleic Acids. 2016 December; 35 (10-12):543-549.
Non-Patent Document 23: Kato S, Kato M, Kusano T, Nishino T. New Strategy That Delays Progression of Amyotrophic Lateral Sclerosis in G1H-G93A Transgenic Mice: Oral Administration of Xanthine Oxidoreductase Inhibitors That Are Not Substrates for the Purine Salvage Pathway. J Neuropathol Exp Neurol. 2016 Dec. 1; 75(12):1124-1144
Non-Patent Document 24: Ouyang J, Parakhia R A, Ochs R S. Metformin activates AMP kinase through inhibition of AMP deaminase. J Biol Chem. 2011 Jan. 7; 286(1):1-11.
Non-Patent Document 25: Norman B, Sabina R L, Jansson E. Regulation of skeletal muscle ATP catabolism by AMPD1 genotype during sprint exercise in asymptomatic subjects. J Appl Physiol (1985). 2001 July; 91(1):258-64.
Non-Patent Document 26: Ogasawara N, Goto H, Yamada Y, Nishigaki I, Itoh T, Hasegawa I, Park K S. Deficiency of AMP deaminase in erythrocytes. Hum Genet. 1987 January; 75(1):15-8.
Non-Patent Document 27: Mineo I, Kono N, Shimizu T, Hara N, Yamada Y, Sumi S, Nonaka K, Tarui S. Excess purine degradation in exercising muscles of patients with glycogen storage disease types V and VII. J Clin Invest. 1985 August; 76(2):556-60.
Non-Patent Document 28: Garrett, R. H. & Grisham, C. M. in Biochemistry 927-956 (Cengage Learning, 2016).

### SUMMARY OF INVENTION

### Technical Problem

The aforementioned symptoms are all symptoms seen in aging. In these papers, the authors mainly focus on mechanism by which xanthine oxidase/xanthine dehydrogenase inhibitors suppress reactive oxygen species. However, the inventors thought that this action was due to inhibition of purine decomposition and energy savings. In other words, it was considered that this action was due to inhibiting the XOR in FIG. 1 and suppressing the decomposition of hypoxanthine. Hypoxanthine is converted to IMP by the action of HGPRT in FIG. 1, and ATP enhancement occurs. If this mechanism is correct, adding inosine to a xanthine oxidase/xanthine dehydrogenase inhibitor can further exert an ATP-enhancing effect. The inventors have already reported in a paper that, comparing with a xanthine oxidase/xanthine dehydrogenase inhibitor alone, adding inosine to a xanthine oxidase/xanthine dehydrogenase inhibitor increases hypoxanthine and enhances ATP (Non-Patent Document 12).

Treatment with a xanthine oxidase/xanthine dehydrogenase inhibitor alone suppresses aging-like heart disease, dementia, and muscle atrophy. Therefore, it was considered that this treatment might suppress aging and prolong lifespan. In addition, since this mechanism is thought to be due to increasing hypoxanthine and enhancing ATP, adding inosine to a xanthine oxidase/xanthine dehydrogenase inhibitor may further suppress aging and prolong lifespan in humans. Research on life extension in humans is not easy. However, it is possible to investigate the effect of life extension by using organisms with a very short lifespan, such as C. elegans.

As will be described later, the metabolic rate of C. elegans, which is a small organism, per weight is much faster than that of humans, and therefore the production rate of hypoxanthine is considered to be much faster. Therefore, in an experiment using C. elegans, only a xanthine oxidase/xanthine dehydrogenase inhibitor was administered without adding inosine to examine whether or not the lifespan was extended.

As mentioned above, mitochondrial disease can be considered as a model of human aging. Therefore, a xanthine oxidase/xanthine dehydrogenase inhibitor and inosine were simultaneously administered to patients with mitochondrial disease to examine whether there is an increase in hypoxanthine and ATP.

Aging and death are one of the greatest tragedies to not only humans but also various organisms such as pets. If this can be prevented or suppressed, it will bring happiness to various organisms such as humans and pets. However, in reality, it is difficult to prevent aging and extend the lifespan.

It has been reported that calorie restriction and administration of metformin, etc. prevent aging and prolong lifespan. However, it is unclear whether the effect is certain and sufficient. A problem to be solved by the present invention is the progress of aging and the limited lifespan of a human or non-human organism. Therefore, an object of the present invention is to enable prevention of aging and extension of lifespan of a human or non-human organism by a method other than calorie restriction and administration of metformin.

### Solution to Problem

The inventors of the present invention have found that by administering an agent comprising A) a xanthine oxidase/xanthine dehydrogenase inhibitor, it is possible to prevent aging and extend the lifespan of a human or non-human organism.

In addition, the inventors of the present invention have found that it is possible to prevent aging and extend the lifespan of a human or non-human organism by combining A) a xanthine oxidase/xanthine dehydrogenase inhibitor and B) hypoxanthine, or a compound that is capable of being converted to hypoxanthine in body, by administering these simultaneously or administering these as a combination drug or a kit formulation, and have completed the present invention.

That is, the present invention has the following configuration.

(1) An anti-aging agent or a life-extending agent comprising the following A):

A) a xanthine oxidase/xanthine dehydrogenase inhibitor.

(2) An anti-aging agent or a life-extending agent comprising a combination of the following A) and B):

A) a xanthine oxidase/xanthine dehydrogenase inhibitor; and

B) hypoxanthine, or a compound that is capable of being converted to hypoxanthine in body.

(3) The anti-aging agent or life-extending agent according to (1) or (2), wherein A) is anyone or more selected from the group consisting of febuxostat, topiroxostat, allopurinol, hydroxyalkane, carprofen, and Y-700.

(4) The anti-aging agent or life-extending agent according to (2) or (3), wherein the compound that is capable of being converted to hypoxanthine in the body of B) is any one or more compounds selected from inosine, inosinic acid, adenosine, AMP, ADP, ATP, succinyl adenosine, S-adenosylmethionine, S-adenosylhomocysteine, and pharmaceutically acceptable salts thereof.

(5) The anti-aging agent or life-extending agent according to any one of (2) to (4), wherein the combination of A) and B) is a combination drug or kit formulation comprising A) and B).

(6) An anti-aging agent or a life-extending agent comprising febuxostat.

(7) An anti-aging agent or a life-extending agent comprising a combination of febuxostat and inosine.

(8) The anti-aging agent or life-extending agent according to (7), wherein the anti-aging agent or life-extending agent is in the form of a combination drug or kit formulation comprising 10 to 80 mg of febuxostat and 0.5 to 4 g of inosine.

(9) Use of febuxostat in combination with inosine for an anti-aging agent or a life-extending agent.

Further, the present invention also includes the following methods.

(101) A method of preventing aging or extending lifespan, comprising a step of administering the following A) to a subject:

A) a xanthine oxidase/xanthine dehydrogenase inhibitor.

(102) A method of preventing aging or extending lifespan, comprising a step of administering the following A) and B) in combination to a subject:

A) a xanthine oxidase/xanthine dehydrogenase inhibitor; and

B) hypoxanthine, or a compound that is capable of being converted to hypoxanthine in body.

(103) The method of preventing aging or extending lifespan according to (101) or (102), wherein A) is any one or more selected from the group consisting of febuxostat, topiroxostat, allopurinol, hydroxyalkane, carprofen, and Y-700.

(104) The method of preventing aging or extending lifespan according to (102) or (103), wherein the compound that is capable of being converted to hypoxanthine in the body of B) is anyone or more compounds selected from inosine, inosinic acid, adenosine, AMP, ADP, ATP, succinyl adenosine, S-adenosylmethionine, S-adenosylhomocysteine, and pharmaceutically acceptable salts thereof.

(105) The method of preventing aging or extending lifespan according to any one of (102) to (104), wherein the combination of A) and B) is a combination drug or kit formulation comprising A) and B).

(106) A method of preventing aging or extending lifespan, comprising a step of administering febuxostat to a subject.

(107) A method of preventing aging or extending lifespan, comprising a step of administering febuxostat and inosine in combination to a subject.

(108) The method of preventing aging or extending lifespan according to (107), wherein the combination of febuxostat and inosine comprises 10 to 80 mg of febuxostat and 0.5 to 4 g of inosine.

(109) The method of preventing aging or extending lifespan according to any one of (101) to (108), wherein the subject is a human or a non-human mammal.

### Advantageous Effects of Invention

According to the present invention, it is possible to prevent aging and extend the lifespan of a human or non-human organism by administering an agent comprising A) a xanthine oxidase/xanthine dehydrogenase inhibitor.

Moreover, it is possible to prevent aging and extend the lifespan of a human or non-human organism by administering the following A) and B) in combination:

A) a xanthine oxidase/xanthine dehydrogenase inhibitor; and

B) hypoxanthine, or a compound that is capable of being converted to hypoxanthine in the body, for example, one or more compounds selected from inosine, inosinic acid, adenosine, AMP, ADP, ATP, succinyl adenosine, S-adenosylmethionine, S-adenosylhomocysteine, and pharmaceutically acceptable salts thereof.

Currently, there have been known effective methods and agents for preventing aging and extending the lifespan of humans or non-human organisms; however, the effects are not sufficient. Therefore, the anti-aging agent or life-extending agent of the present invention is extremely useful.

### BRIEF DESCRIPTION OF DRAWINGS

FIG. 1 is a diagram showing of the pathway of purine metabolism. In the figure, de novo synthesis of purine nucleotide begins with ribose-5-phosphate, which is obtained from pentose monophosphate shunt from the glycolytic pathway. IMP is synthesized through a series of enzymatic reactions in which 6 ATPs are used. In one of the reactions in which 6 ATPs are used, AMP, rather than ADP, is generated, and thus actually 7 molecules of ATP are consumed. When ATP is consumed by muscular movement and the like and becomes ADP, ADP is converted to ATP by glycolysis or oxidative phosphorylation in mitochondria. However, two molecules of ADP can also be converted to ATP and AMP by the action of AK (adenylate kinase). This reaction synthesizes ATP without an involvement of glycolysis or oxidative phosphorylation. However, as a by-product, AMP is produced, which reduces energy charge. To avoid this, cells convert AMP to IMP by AMPD (AMP deaminase) and to hypoxanthine and finally to uric acid by xanthine oxidoreductase. At the hypoxanthine stage, it can be converted back to IMP by the action of HGPRT, and finally converted to ATP. However, when it is decomposed by xanthine oxidase/xanthine dehydrogenase, it cannot be converted back to purine nucleotide, and 7 molecules of ATP must be newly consumed for synthesis.

FIG. 2 is a graph showing an effect of febuxostat (FBX) on nematode lifespan using an N2 strain of C. elegans. Kaplan-Meier curves in an experiment in which 50 nematodes were observed per condition are shown. All P values are based on a log-rank (Mantel-Cox) test.

FIG. 3 is a graph showing an effect of febuxostat (FBX) on nematode lifespan using a daf-16 gene variant strain of C. elegans. Kaplan-Meier curves in an experiment in which 50 nematodes were observed per condition are shown. All P values are based on a log-rank (Mantel-Cox) test.

FIG. 4 is a graph showing an effect of febuxostat (FBX) on nematode lifespan using a wild strain N2 of C. elegans. Kaplan-Meier curves in an experiment in which 50 nematodes were observed per condition are shown.

FIG. 5 shows a result of an electron microscopic observation of inside of muscles in the tail of nematode fixed after culturing nematode in the absence (FBX(−)) or in the presence (20 μg/mL FBX) of febuxostat (FBX) for 18 days.

### DESCRIPTION OF EMBODIMENTS

### (Anti-Aging Agent or Life-Extending Agent)

The anti-aging agent or life-extending agent of the present invention refers to an agent for suppressing aging (advancement in age) and extending the lifespan of a human or non-human organism. The agent of the present invention can be used as a pharmaceutical product.

Non-human organisms include invertebrates, and vertebrates such as birds, fish, amphibians, reptiles, and mammals, especially mammals such as cows, canines, horses, cats, sheep, pigs, rabbits and mice.

Regarding the confirmation of the life-extending effect, it has been reported that the anti-diabetes drug metformin prolongs the lifespan in C. elegans (Non-Patent Documents 13 to 16), and it has been suggested that the same drug prevents aging and prevents death from various causes in Drosophila (Non-Patent Document 17) or in humans (Non-Patent Document 18). Therefore, the life-extending effect shown in C. elegans can also be expected in humans and non-human organisms.

Larger mammals have a lower energy metabolism rate per weight than that of smaller mammals. In general, the energy metabolism rate of an individual mammal is proportional to the ¾ power of body weight according to Kleiber's law (Non-Patent Documents 19 and 20), so the energy metabolism rate per weight is proportional to −¼ power of body weight. For example, human energy metabolism (per weight) is considered to be about ⅛ that of mice. The energy consumed by mammals per weight for a lifetime is almost constant. Therefore, it is considered that larger mammals consume less energy per weight and per hour, and thus have a long lifespan (Non-Patent Document 20). Since most of the energy in the body of an organism is consumed as ATP, the energy consumption is considered to be almost the same as the consumption of ATP. Uric acid production is associated with ATP consumption, and high ATP consumption tends to increase the rate of uric acid production (Non-Patent Document 21). Since ATP consumption in mice is significantly faster than that in humans, the rate of uric acid production in mice is about 25 times that in humans (Non-Patent Document 22). Therefore, it has been known that in mice, a xanthine oxidase/xanthine dehydrogenase inhibitor alone shows a significant increase in blood hypoxanthine (Non-Patent Document 23), while in humans there is only a slight increase, and simultaneous administration of inosine significantly increases hypoxanthine (Non-Patent Document 12). Therefore, especially in large organisms such as humans, comparing with administering a xanthine oxidase/xanthine dehydrogenase inhibitor alone, simultaneous administration of a compound that is capable of being converted to hypoxanthine in the body, such as inosine, with a xanthine oxidase/xanthine dehydrogenase inhibitor may further strongly prevent aging and extend lifespan (Non-Patent Document 12).

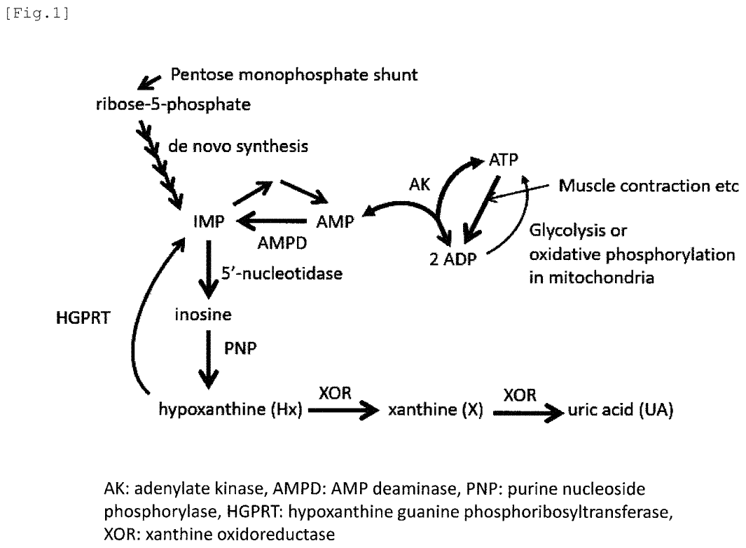

Activation of AMP activated protein kinase (AMPK) is considered as a mechanism of life extension by metformin (Non-Patent Document 13). It is considered that metformin accumulates AMP by suppressing AMP deaminase (AMPD), which may activate AMPK (FIG. 1) (Non-Patent Document 24). AMPK is thought to have an effect of detecting the accumulation of AMP, suppressing the consumption of ATP and promoting the production in order to maintain the energy state of cells. It has been reported that in human genetic AMPD deficiency, AMP accumulation occurs and ATP enhancement (or suppression of decrease) occurs (FIG. 1). That is, three genes encoding human AMPD are known, AMPD1, AMPD2, and AMPD3, and deficiencies comprising homozygotes have been reported for each. However, it has been reported that ATP of muscles (Non-Patent Document 25) and erythrocytes (Non-Patent Document 26) is enhanced in homozygotes with AMPD1 and AMPD3 deficiency, respectively. In the former, the decrease in ATP on motion is suppressed, and in the latter, the amount of ATP in erythrocytes increases. When ATP is consumed rapidly, ATP becomes short and ADP accumulates (FIG. 1). In this state, AK acts in a direction of synthesizing AMP and ATP from two molecules of ADP, and AMP accumulates. The accumulated AMP is decomposed into IMP by AMPD and finally converted to uric acid by xanthine oxidase/xanthine dehydrogenase (FIG. 1). It has been reported that a serum uric acid level rises in a state of energy crisis where ATP is actually consumed rapidly (Non-Patent Documents 21 and 27). When it is decomposed by xanthine oxidase/xanthine dehydrogenase and becomes xanthine and uric acid, it is impossible to return to nucleotides; however, in the state of hypoxanthine, it is possible to return to nucleotides by the action of HGPRT (FIG. 1). In order to regenerate purine nucleotides lost as xanthine and uric acid, it is necessary to synthesize IMP from ribose-5-phosphate obtained from the pentose monophosphate shunt (purine de novo synthesis), and this requires consuming 7 molecules of ATP (Non-Patent Document 28) (FIG. 1).

The xanthine oxidase/xanthine dehydrogenase inhibitor has effects of inhibiting the xanthine oxidase/xanthine dehydrogenase shown in FIG. 1, suppressing the decomposition of purines, and suppressing the decrease of purine nucleotides through HGPRT (FIG. 1). That is, it is considered to increase hypoxanthine, increase IMP through HGPRT, and further increase AMP and ATP (Non-Patent Document 12). In situations where ATP is used rapidly and decreases, the decrease is suppressed. Metformin also has an effect of suppressing the decrease in purine nucleotides through the inhibition of AMPD (FIG. 1). That is, there may be something in common between the life-extending effect by administrating of febuxostat or simultaneous administrating of febuxostat and inosine, and the life-extending effect of metformin.

### (Active Ingredient)

One active ingredient of the present invention is A) a xanthine oxidase/xanthine dehydrogenase inhibitor. Examples of the xanthine oxidase/xanthine dehydrogenase inhibitor include febuxostat (trade name: Feburic), topiroxostat (FUJIYAKUHIN), allopurinol (trade name: Zyloric, Alositol, etc.), hydroxyalkane, carprofen, and Y-700 (Mitsubishi Tanabe Pharma), among which febuxostat is desirable. The anti-aging agent or life-extending agent of the present invention may comprise this active ingredient alone, as long as this active ingredient is comprised.

Another active ingredient of the present invention is B) hypoxanthine, or a compound that is capable of being converted to hypoxanthine in the body, for example, one or more compounds selected from inosine, inosinic acid, adenosine, AMP, ADP, ATP, succinyl adenosine, S-adenosylmethionine, S-adenosylhomocysteine, and pharmaceutically acceptable salts thereof. Among them, inosine is desirable. The anti-aging agent or life-extending agent of the present invention preferably comprises this active ingredient in addition to the active ingredient of A) above.

### (Combination)

“Comprising a combination of A) and B)” of the present invention is used to mean all the forms in which the components A) and B) are combined. Therefore, this includes either a combination drug of the components A) and B) mixed to form a composition, or drugs presented together to be administered in the same period at the time of administration although physically present separately without being mixed. Examples of the combination drug of the components A) and B) mixed to form a composition include those mixed as a drug formulation. Examples of drug formulation include oral agents such as granules, powders, solid formulations, and liquids, and suppositories. Examples of the drugs presented together to be administered in the same period at the time of administration although physically present separately include a so-called kit formulation and a form of being packed in one bag. The same period does not necessarily mean the same time in a strict sense, and the same period of the present invention includes the case that an interval exists within a range in which the effect is exerted. For example, in the case of an oral agent, when one is taken before meal and the other is taken after meal, this corresponds to the case of administering in the same period of the present invention.

As described above, it is also possible to take A) and B) as individual formulations. However, taking B) alone may be harmful because it causes hyperuricemia, and A) and B) must be taken in the same period. From this point, a kit formulation or a combination drug is desirable, and a combination drug is even more desirable.

An invention of an anti-aging agent or a life-extending agent comprising a combination of A) and B) of the present invention is, in other words, an invention of a method of preventing aging, or a method of extending lifespan, comprising a step of administering the aforementioned A) and B) in combination. The timing of administration of each is as described above.

### (Dosage)

The dosages of the agents of the present invention may be any effective amount, and the following respective dosages are desirable. For example, it is desirably 10 to 80 mg/day for febuxostat, 40 to 160 mg/day for topiroxostat, and about 50 mg to about 800 mg/day for allopurinol of A). Further, it is desirably 0.5 to 4.0 g/day for inosine of B), and the effective amount of B) hypoxanthine, or a compound that is capable of being converted to hypoxanthine in the body can also be determined by converting an amount corresponding to the amount of inosine by the molecular weight.

### (Administration Method)

With regard to an administration method, each of the dosages can be administered once a day, or divided twice or more a day. Among them, febuxostat is desirably administered twice a day rather than once a day as in a conventional usage of febuxostat. It is also desirable that inosine is administered twice a day rather than once a day. Therefore, both inosine and febuxostat are more desirably divided and administered twice a day.

In the case of a combination drug, adjustment may be made in consideration of a daily dosage and an administration method, and febuxostat and inosine may desirably be adjusted by adding 0.5 g, 1 g, 1.5 g, or 2 g of inosine to 20 mg or 40 mg of febuxostat. It is more desirable to add 20 mg of febuxostat and 0.5 g of inosine to one table.

### (Administration Form)

The administration form of the drug of the present invention is not particularly limited, and either oral or parenteral administration form may be used. In addition, it can be made into an appropriate dosage form according to the administration form. For example, it can be prepared into various formulations such as injections; oral agents such as capsules, tablets, granules, powders, pills, and fine granules; rectal administration agents, oily suppositories, and aqueous suppositories.

Among the agents of the present invention, for the administration form of an agent comprising the active ingredients A) and B), the administration form of the active ingredient A) and the administration form of B) may be the same or different. Examples of the same administration form include a case where both are orally administered as tablets, a case where both are orally administered as a combination drug, and a case where both are administered as a mixed injection. Further, examples of different administration forms include a case where one is administered as an oral agent and the other is administered as an injection.

(Combination Use with Conventional Anti-Aging Agents or Life-Extending Agents)

The anti-aging agent or life-extending agent of the present invention exerts an effect of enhancing intracellular ATP, and can be used in combination with an existing anti-aging agent or life-extending agent already prescribed within a range not to impair the action of the present invention.

The effects of the anti-aging agent or life-extending agent of the present invention will now specifically be described based on examples; however, the present invention is not limited thereto.

### EXAMPLES

### Example 1

Examination of Extension of C. elegans (Nematode) Lifespan by Febuxostat

Extension of lifespan by febuxostat was examined using a wild type strain (N2, Bristrol) and a daf-16 gene (nematode ortholog of human transcription factor FOXO; a typical example of shortened lifespan in nematode variant due to a transcription factor that is suppressed by insulin signals) variant strain (mu86). Both strains were bred at 20° C.

### 1. Test Method

### (1) Preparation in Advance

In order to keep the developmental stage of the strains used for lifespan measurement constant, it was waited until food (Escherichia coli) was exhausted and growth of first instar larvae stopped. The nematode was cut and planted together with agar in a petri dish with food.

### (2) The Day Before

Ampicillin (100 μg/ml) was added to a 6 cm NGM plate (standard medium for nematode culture) to inhibit the growth of the food Escherichia coli strain and its associated metabolism, and OP50 (the food Escherichia coli strain) and FBX were added (FBX concentration 0, 10, 20 or 40 mg/L).

### (3) Day 0

When the cut and planted nematodes became adults, they were washed out of the petri dish using M9 buffer and collected. The nematodes were suspended in Eppendorf tubes. An equal amount of bleach solution (a mixture of 4N NaOH, hypochlorous acid solution, and M9 buffer at a ratio of 2:3:5) to the nematode suspension was added and mixed. The supernatant was discarded by centrifugation at 400×g. Adults ruptured and died, and only fertilized eggs were recovered alive. The eggs were placed on the NGM plate (with ampicillin, with/without FBX) prepared the day before, and the culture was started.

### (4) Day 4

FUdR (final concentration 0.5 mg/ml) was added to the medium when the bleached eggs grew to a period where egg-laying became possible. This eliminated the effect of spawning on lifespan by making adults infertile.

### (5) Day 5

A petri dish in which FUdR (final concentration 0.5 mg/ml) was added to a 35 mm NGM petri dish comprising ampicillin and febuxostat after applying Escherichia coli in advance was prepared in the same manner as in (4) above. Five adults were placed on one petri dish and breeding was started. For each sample, 10 or more petri dishes were simultaneously subjected to observation in one experiment.

### (6) After Day 6

Life or death was determined for each individual every other day, and the data was recorded. A life/death determination method at this time was carried as follows. “A moving nematode or an individual whose pharyngeal muscles are pumping to ingest food is considered alive. When there was a non-moving nematode, it was gently touched with a platinum wire (nematode picker) to observe whether it would react.”

### (7) End of Observation

The lifespan tracking was completed when it was determined that all nematodes had died.

### 2. Result

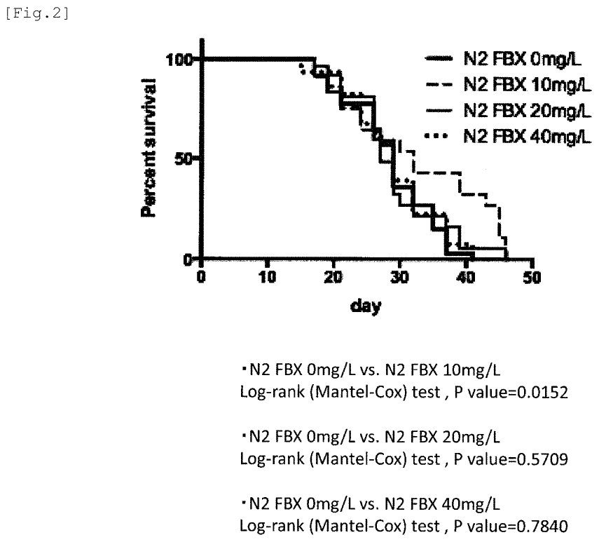

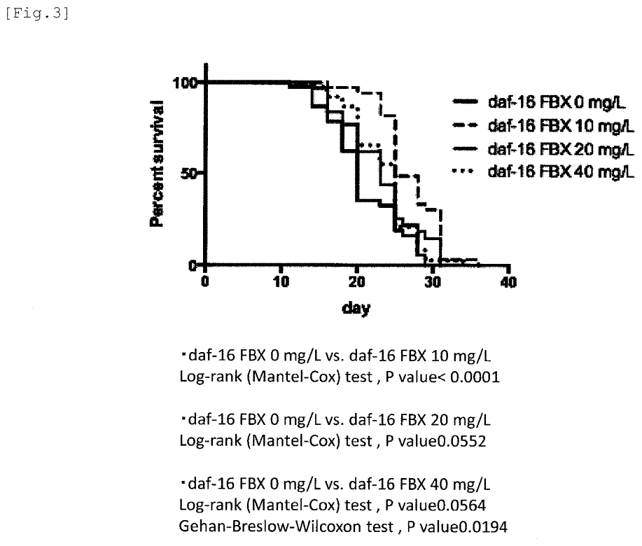

Both the wild type strain (N2, Bristrol) (FIG. 2) and the daf-16 gene variant strain (mu86) (FIG. 3) showed an extension of lifespan in the presence of febuxostat 10 mg/ml; however, the daf-16 gene variant strain (mu86) did not reach the same length of lifespan as that of the wild type strain even febuxostat was administered. Therefore, it was speculated that the action of febuxostat works in a different pathway from the daf-16 gene.

### Example 2

Examination of Increase in Hypoxanthine and ATP by Simultaneous Administration of Febuxostat and Inosine in Patients with Mitochondrial Disease

Mitochondrial disease is definitely associated with the etiology of ATP deficiency. Therefore, it was investigated whether simultaneous administration of febuxostat, which is a xanthine oxidase/xanthine dehydrogenase inhibitor, and inosine would actually increase ATP in patients with confirmed mitochondrial disease.

### 1. Test Method

The case is an 80-year-old male with a diagnosis of mitochondrial cardiomyopathy. Severe heart failure and multiple premature ventricular contractions (PVC) were detected at the age 60. Direct sequencing of mitochondrial DNA (mtDNA) from blood revealed a homoplasmy mutation (m.12192G>A) in the histidine mitochondrial tRNA gene (MT-TH), and thus it was diagnosed as mitochondrial cardiomyopathy.

Febuxostat 20 mg and inosine 0.5 g were administered to the patient twice daily for 14 days.

### 2. Measurement Method of Purines (ATP, Hypoxanthine)

Various purines in peripheral blood were measured according to the following reference document.

Briefly, peripheral blood was collected in a test tube comprising EDTA-2Na, 500 μL of blood was mixed with 500 μL of ice cold 8% PCA, and the mixture was immediately vortexed. Then, the mixture was centrifuged at 12,000×g for 5 seconds at 4° C., and the supernatant was stored at −80° C. 40 μL of 2M K₂CO₃ in 6M KOH was added to 650 μL of the above specimen solution to precipitate PCA and neutralize the solution at the same time. After this solution was centrifuged at 12,000×g for 10 minutes at 4° C., 160 μL of a mobile phase was added to 40 μL of the supernatant and apply to HPLC. The conditions of HPLC were also set according to Reference 1 below. The amounts of purines are expressed in molar quantity contained in the solution after PCA was removed.

### [Reference Document 1]

Kamatani N, Hashimoto M, Sakurai K, Gokita K, Yoshihara J, Sekine M, Mochii M, Fukuuchi T, Yamaoka N, Kaneko K. Clinical studies on changes in purine compounds in blood and urine by the simultaneous administration of febuxostat and inosine, or by single administration of each. Gout and Nucleic Acid Metabolism 41, 171-181, 2017.

### 3. Result

Serum uric acid value, BNP value, ATP amount in blood, hypoxanthine amount and xanthine amount were measured before and after treatment. It was confirmed that the serum uric acid value was 1.29 mg/dL from 3.04 mg/dL and the BNP value was 204.4 pg/mL from 295.1 pg/mL, which was a 31% decrease. In an electrocardiogram, PVC disappeared, while atrial fibrillation remained. In addition, systolic blood pressure and diastolic blood pressure decreased without adverse events.

Furthermore, as shown in Table 1, the ATP amount in blood increased 1.3 times, the amount of hypoxanthine increased 6.5 times, and the amount of xanthine increased 19.1 times.

Therefore, it was confirmed that the simultaneous administration of febuxostat, which is one of the xanthine oxidase/xanthine dehydrogenase inhibitors, and inosine, which is capable of being converted into hypoxanthine in the body, increases hypoxanthine and ATP in the blood.

TABLE 1

| | Before treatment | After treatment |
|---|---|---|
| ATP* | 175.0 | 224.7 |
| Hypoxanthine* | 2.6 | 16.8 |
| Xanthine* | 0.8 | 15.3 |

*Each value indicates the concentration in the final solution of the measurement test.

### Example 3

Examination of Effect of Febuxostat on C. elegans (Nematode) Lifespan and Mitochondrial Morphological Change

The effect of febuxostat on lifespan was examined using a wild type strain (N2, Bristrol) of C. elegans as a nematode. In addition, mitochondria of the nematode cultured in the absence and presence of febuxostat were compared under electron microscopy.

### 1. Test Method

### (1) Effect of Febuxostat on Nematode Lifespan

The culturing method and culturing conditions for nematodes (N2) were almost the same as those in Example 1 except for the method for treating the food added to the medium. Generally, Escherichia coli is added to the NGM plate as food for nematodes; however, when Escherichia coli grows, it may affect metabolites, etc. Therefore, a method of adding the antibiotic ampicillin to stop the growth of Escherichia coli is performed. However, it cannot be completely denied that the presence of ampicillin in the NGM plate had some effect.

Therefore, instead of adding ampicillin, Escherichia coli was killed by being irradiated with ultraviolet rays, and kanamycin was further added. In this way, Escherichia coli as food was treated differently from Example 1, and it was checked whether the same experimental results as in Example 1 could be obtained.

The concentration of febuxostat added to the NGM plate was 5, 10, 20 μg/mL.

### (2) Effect of Febuxostat on Mitochondrial Morphology in Nematode Muscle

On the 18th day of culturing, nematodes were fixed by the following method. An M9 buffer comprising 8% ethanol was dropped on a slide glass, and the nematodes were placed therein and allowed to stand until the nematodes stopped moving (about 5 minutes). Next, a fixing solution was placed on another slide glass, and the nematodes that had stopped moving were transferred to the fixing solution. The fixing solution comprises 2% paraformaldehyde+2% glutaraldehyde/0.1M phosphate buffer (pH 7.4), and was adjusted prior to use.

Subsequently, a 22 G injection needle was used to cut the nematodes in the following procedure.

First, the cranial side was cut from the pharynx, and then amputation was performed around Vulva. In this experiment, in order to observe the mitochondria of the muscle behind Vulva with an electron microscope, only the caudal side was collected and placed in an Eppendorf tube comprising a fixing solution (4° C.)

Observation with an electron microscope was carried out by requesting the Hanaichi UltraStructure Research Institute (Okazaki City, Aichi Prefecture) using an electron microscope HITACHI H-7600 (100 kV).

### 2. Result

### (1) Effect of Febuxostat on Nematode Lifespan

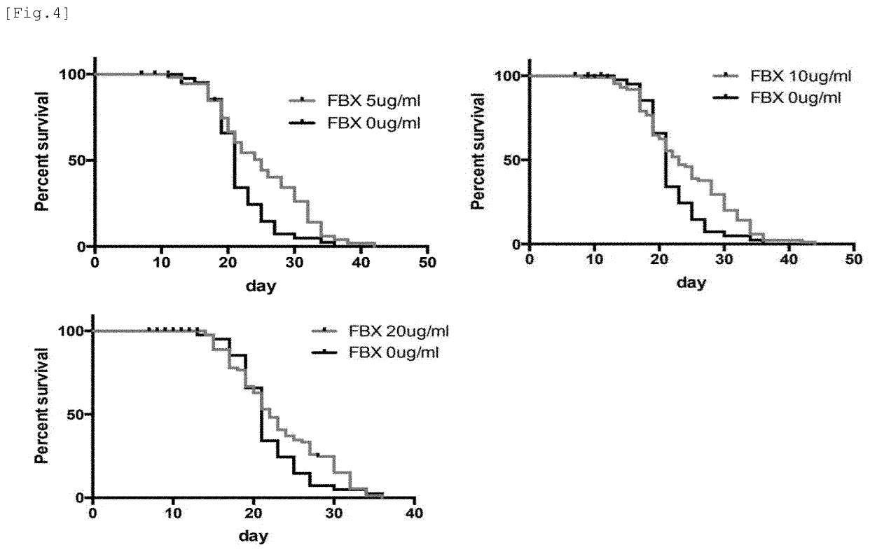

As shown in FIG. 4, it was shown that the lifespan of nematodes is extended by adding febuxostat. This result was almost the same as that of Example 1, indicating that febuxostat extends the lifespan of nematodes.

### (2) Effect of Febuxostat on Mitochondrial Morphology in Nematode Muscle

Nematode slows down as it approaches death, partly because the mitochondria of the muscle are damaged and muscle strength is weakened.

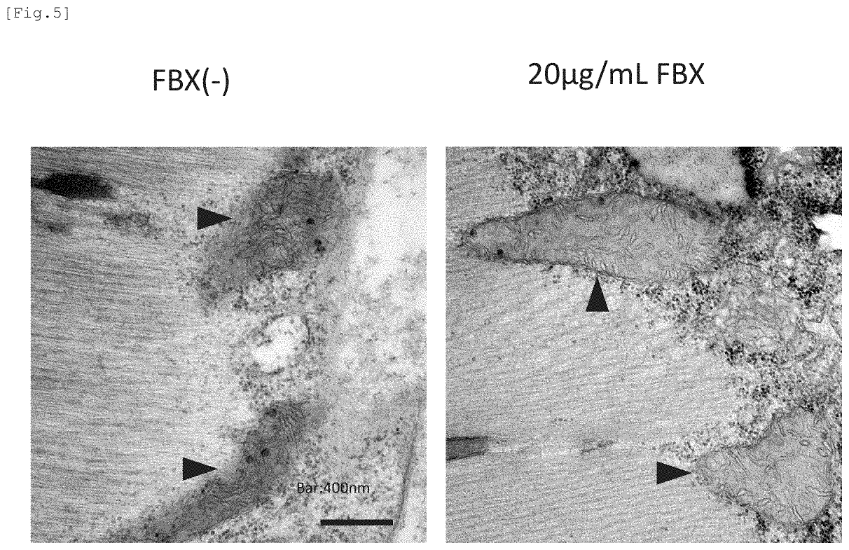

An electron micrograph in the muscle of a nematode cultured for 18 days without adding febuxostat is shown in FIG. 5 “FBX(−)”. Two mitochondria were confirmed in this figure; however, their normal morphology was destroyed. That is, the structure of the outer and inner membranes of mitochondria was not clearly seen, and although part of cristae could be confirmed, the shape was broken (black arrow in the photograph of FIG. 5).

FIG. 5 “20 μg/mL FBX” shows an electron micrograph of mitochondria in the muscle of the same nematode cultured for the same 18 days in the presence of 20 μg/mL of febuxostat. Comparing with FIG. 5 “FBX(−)”, the structures of inner membrane, outer membranes, and cristae, etc. were clearly confirmed (black arrow in the photograph of FIG. 5). That is, it is shown that the morphology of mitochondria is maintained almost normally.

### 3. Discussion

It has already been shown in Example 1 that febuxostat extends the lifespan of nematodes. That is, it has been shown that the lifespan was extended in the presence of febuxostat using two nematode strains, a wild type strain (N2, Bristol) and a daf-16 gene variant strain (mu86).

Further, in Example 2, it was shown that febuxostat, and inosine which produces hypoxanthine in the body, are simultaneously administered to patients with mitochondrial disease to increase hypoxanthine and ATP in blood. Furthermore, it has already been announced that simultaneous administration of febuxostat, and inosine which produces hypoxanthine in the body, dramatically improved the disease biomarkers BNP and insulin index in patients with mitochondrial disease (see Reference Document 2 below).

In Example 3 above, the life-extending effect of febuxostat using a wild type strain (N2) of nematodes was reconfirmed, and it was further shown that febuxostat suppresses morphological changes due to aging of intramuscular mitochondria of aged nematodes.

Mitochondria are intracellular organelles that regenerate ATP, and by integrating the above examples, it was shown that mitochondrial deterioration is very likely to be associated with aging and longevity of organisms. It was also shown that febuxostat suppresses mitochondrial deterioration. It has been shown by a large amount of conventional data that the action of febuxostat is due to the inhibition of xanthine oxidoreductase. In general, inhibition of xanthine oxidoreductase can prevent the deterioration of nematode mitochondria, and thus it is presumed that aging can be suppressed and the life span can be extended. On the other hand, based on the experimental results that metformin extends the lifespan of nematodes, experiments were carried out on Drosophila and mice and similar results were obtained. The results suggest that human aging is prevented and the lifespan is extended, and large-scale clinical trials are planned. Combining a series of experiments conducted with metformin and the trial plans, it is believed that xanthine oxidoreductase inhibitors generally prevent aging and extend lifespan of organisms according to an integration of the data revealed in the present application. The effect of xanthine oxidoreductase inhibitors is thought to be based on enhancing ATP; however, the differences between species in purine metabolism are significant, and the uric acid production rate in humans is only 1/25 of that in mice. It is thought that this is because the size of an organism and the energy consumption are inversely proportional. And as mentioned above, the effect of xanthine oxidoreductase inhibitors alone is considered to be weak in humans and large organisms, and it is more desirable to simultaneously administer hypoxanthine, or a substance that produces hypoxanthine, such as inosine, in the body.

### [Reference Document 2]

Kamatani N, Kushiyama A, Toyo-Oka L, Toyo-Oka T. Treatment of two mitochondrial disease patients with a combination of febuxostat and inosine that enhances cellular ATP. J Hum Genet. 2019 April; 64(4):351-353.

### [Formulation Example 1] Example of Combination Drug

A combination drug (tablet type) for oral administration was manufactured, having the following contents per tablet.

Febuxostat: 20 mg

Inosine: 0.5 g

Alpha starch (disintegrating binder): 70 mg

Silicifited microcrystalline cellulose (filler): 32.656 mg

Croscarmellose sodium (disintegrant): 10 mg

Magnesium Stearate (lubricant): 0.8 mg

### [Formulation Example 2] Example of Kit Formulation

Tablets having the composition of A below containing febuxostat and an agent having the composition of B below containing inosine were put in the same bag separated to prevent mixing with each other for adjustment into one dose. A kit formulation was manufactured by packing two doses, i.e., a daily dosage, thereof in the same box.

### A. Febuxostat Tablets

Febuxostat: 20 mg

Alpha starch (disintegrating binder): 70 mg

Silicified microcrystalline cellulose (filler): 32.656 mg

Croscarmellose sodium (disintegrant): 10 mg

Magnesium stearate (lubricant): 0.8 mg

### B. Inosine

Inosine: 0.5 g

### INDUSTRIAL APPLICABILITY

According to the present invention, it is possible to prevent aging and extend the lifespan of a human or non-human organism by administering an agent comprising A) a xanthine oxidase/xanthine dehydrogenase inhibitor.

Moreover, it is possible to prevent aging and extend the lifespan of a human or non-human organism by administering the following A) and B) in combination:

A) a xanthine oxidase/xanthine dehydrogenase inhibitor; and

B) hypoxanthine, or a compound that is capable of being converted to hypoxanthine in the body, for example, one or more compounds selected from inosine, inosinic acid, adenosine, AMP, ADP, ATP, succinyl adenosine, S-adenosylmethionine, S-adenosylhomocysteine, and pharmaceutically acceptable salts thereof.

1. A method for suppressing aging or extending lifespan comprising: administering to a subject an anti-aging agent or a life-extending agent comprising the following A):
A) a xanthine oxidase/xanthine dehydrogenase inhibitor.

2. A method for suppressing aging or extending lifespan comprising: administering to a subject an anti-aging agent or a life-extending agent comprising a combination of the following A) and B):
A) a xanthine oxidase/xanthine dehydrogenase inhibitor; and
B) hypoxanthine, or a compound that is capable of being converted to hypoxanthine in body.

3. The method of claim 1, wherein A) is any one or more selected from the group consisting of febuxostat, topiroxostat, allopurinol, hydroxyalkane, carprofen, and Y-700.

4. The method of claim 2, wherein a compound that is capable of being converted to hypoxanthine in the body of B) is any one or more compounds selected from inosine, inosinic acid, adenosine, AMP, ADP, ATP, succinyl adenosine, S-adenosylmethionine, S-adenosylhomocysteine, and pharmaceutically acceptable salts thereof.

5. The method of claim 2, wherein the combination of A) and B) is provided as a combination drug or kit formulation comprising A) and B).

6. The method of claim 1, wherein said anti-aging agent or a life-extending agent comprises febuxostat.

7. The method of claim 2, wherein said anti-aging agent or a life-extending agent comprises a combination of febuxostat and inosine.

8. The method of claim 7, wherein the anti-aging agent or life-extending agent is provided in the form of a combination drug or kit formulation comprising 10 to 80 mg of febuxostat and 0.5 to 4 g of inosine.

9. A method for suppressing aging or extending lifespan comprising: administering to a subject febuxostat in combination with inosine for an anti-aging agent or a life-extending agent.

10. The method for suppressing aging or extending lifespan of claim 2, wherein A) is any one or more selected from the group consisting of febuxostat, topiroxostat, allopurinol, hydroxyalkane, carprofen, and Y-700.

11. A method of claim 7, wherein the anti-aging or life-extending agent is administered to a subject with mitochondrial disease.

12. The method of claim 8, wherein the anti-aging agent or life-extending agent is administered daily for a plurality of consecutive days.

13. The method of claim 12, wherein said consecutive days comprises 14 days.

14. The method of claim 7, wherein said subject is a human that is at least 60 years old.

15. The method of claim 11, wherein said mitochondrial disease is mitochondrial cardiomyopathy.
